(19) United States
(12) Patent Application Publication
(10) Pub. No.: US 2023/0102034 A1
(43) Pub. Date: Mar. 30, 2023

(54) SCENARIO DEVELOPMENT FOR AN INCIDENT EXERCISE

(71) Applicant: Lawrence Livermore National Security, LLC, Livermore, CA (US)

(72) Inventors: Gregory K. White, Livermore, CA (US); Steven A. Kreek, Livermore, CA (US)

(21) Appl. No.: 17/489,752

(22) Filed: Sep. 29, 2021

(51) Int. Cl. G06Q 50/26

(52) U.S. Cl. CPC G06Q 50/26

(57) ABSTRACT

An incident exercise system supports an incident exercise. The incident exercise system accesses an exercise plan defining an incident within a theater of operation. The incident exercise system administers the incident exercise by generating detector signals factoring in incident effects and characteristic effects of the theater of operation. The incident exercise system sends detector signals to detectors that filter effect of background noise from the detector signals to generate filtered detector signals and output data derived from the filtered detector signals. The incident exercise system supports dynamically modifying the exercise plan during the incident exercise.

### STATEMENT REGARDING FEDERALLY SPONSORED RESEARCH

This invention was made with Government support under Contract No. DE-AC52-07NA27344 awarded by the United States Department of Energy. The Government has certain rights in the invention.

### BACKGROUND

Many types of materials represent a significant hazard to people, equipment, buildings, and so on. Such hazardous materials may include biological, chemical, and radioactive materials, among others. When a hazardous material incident occurs, an effective response is needed to minimize the harmful effects of the incident. For example, when a hazardous biological material is released into the atmosphere, the first indications of the incident may be reports by people of unusual bodily conditions (e.g., watery eyes and difficulty breathing). When first responders (e.g., members of a hazmat team) arrive at the scene of an incident, the first responders seek to identify the hazardous material. The first responders may have various detectors available to assist in identifying the hazardous material. These detectors may include spectrometers, radiation detectors, seismometers, chemical agent detectors, and so on.

Although the detectors for detecting hazardous materials can be effective at detecting the presence of hazardous materials, the detectors can be complex devices whose effective use may require a significant amount of training. In addition, some detectors may display graphs representing characteristics of measurements and leave it up to a person to interpret the graphs as part of identifying the hazardous materials and non-hazardous material that are present. The proper interpretation of a graph may require training. Although some training may be done in a classroom environment, the most effective training can occur in a field exercise environment with the actual hazardous materials. The use of the actual hazardous material, however, can present many problems such as exposing the participants in the exercise (or the general public) to the hazardous material (e.g., radiation or nerve gas), causing long-lasting contamination to the area of the field exercise, down-wind contamination, and so on. In addition, it is impractical to conduct incident exercises that cover a wide variety of scenarios needed for training. For example, it would be difficult to provide a real-life scenario cover many different types of hazardous material, different amounts of the hazardous material, different types of shielding (e.g., material and thickness), and so on. It would also be difficult to include with non-hazardous uses of hazardous materials that may be commonly used. Such commonly non-hazardous material may be radiation sources of a medical facility, a manufacturing environment, a security checkpoint, a person after radiation therapy, and so on.

### BRIEF DESCRIPTION OF THE DRAWINGS

FIG. 1 is a flow diagram that illustrates the processing of an administer incident exercise component of the incident exercise system in some embodiments.

FIG. 2 is a block diagram that illustrates components of the incident exercise system in some embodiments.

FIG. 3 is a flow diagram that illustrates the processing of a generate detector signals component of the incident exercise system in some embodiments.

FIG. 4 is a flow diagram that illustrates the processing of a generate incident effects component of the incident exercise system in some embodiments.

FIG. 5 is a flow diagram that illustrates the processing of a create exercise plan component of the incident exercise system in some embodiments.

FIG. 6 is a flow diagram that illustrates the processing of a modify exercise plan manually component of the incident exercise system in some embodiments.

FIG. 7 is a flow diagram that illustrates the processing of a modify plan based on images component of the incident exercise system in some embodiments.

### DETAILED DESCRIPTION

Methods and systems for administering an incident exercise based on an exercise plan are provided. In some embodiments, an incident exercise system administers an incident exercise based on an exercise plan that defines the incident exercise. The exercise plan may specify a theater of operation, the type of incidents within the theater of operation, characteristics of objects within the theater of operation (e.g., location, dimensions, and type, and amount of background radiation emitted by the object), detectors for detecting effects of the incidents, the entities (e.g., trainees) participating in the incident exercise, and so on. A theater of operation may be a training facility that includes roads, buildings, and vehicles that provides a variety of environments that may be encountered when an incident occurs. The environments may include, for example, a shipping facility, a school, a governmental office building, a shopping mall, a sports stadium, and a subway station. A theater of operation may also be a non-public facility such as a cruise ship without passengers, a container shipping facility, a military base, a closed airport terminal, and so on. Although the theater of operation could be a location that is currently publicly accessible (e.g., the National Mall area in Washington D.C.), the public's reaction to the incident exercise (e.g., panic) would need to be considered in deciding on the location.

In some embodiments, an exercise plan specifies hazardous material incidents by indicating the type and amount of a hazardous material, the location (e.g., on land, underwater, in the atmosphere) and timing of the hazardous material incident, and so on. The incident exercise system may support of variety of types of hazardous material incidents such as an explosion of a dirty bomb, release of toxic gases (e.g., sarin gas), a malfunction with a non-hazardous use (e.g., at a medical facility), toxic chemical in a water supply system, and so on.

In some embodiments, the exercise plan specifies the objects within the theater of operation such as buildings, roads, non-hazardous uses (of a hazardous material), vehicles (moving or stationary), industrial facilities, medical facilities, bridges, vegetation, topography, groups of people, a landfill that emits a gas (e.g., hydrogen sulfide) or other observables, a facility with equipment that may emit exercise-relevant signals (e.g., radiation) that is well above the amount that occurs naturally, and so on. The exercise plan also specifies characteristics of the objects such as object types (e.g., building, train, or landfill), locations, dimensions, compositions (e.g., asphalt, cement, dirt, and water), content (e.g., nuts, stones, or fertilizers within a shipping container), and so on. The characteristics also include amount and type of radiation emitted by an object (e.g., a building or a medical device), the amount of shielding of radiation provided by the object (e.g., a lead wall), the amount and type of gas released by an object (e.g., a landfill), and so on.

The exercise plan specifies participants and detectors of an incident exercise. The participants may be assigned roles such as trainee or terrorist. The detectors may be specified by brand and model number, type (e.g., chemical detectors and radiation detectors), and so on. The exercise plan also specifies locations of stationary detectors and the entity (e.g., training or drone) associated with a non-stationary detector. The incident exercise system may include software components for different types of detectors that generate detector signals based on the exercise plan, location of detectors, and so on.

The incident exercise system implements an exercise plan by determining locations of detectors and calculating incident effects of hazardous materials at detector locations within the theater of operation at the current time. For example, if an incident is the release of a toxic gas at a designated location, then the incident exercise system may calculate incident effects based on a model of the dispersal of the toxic gas at various locations considering factors such as amount of the toxic gas, the current weather conditions (e.g., temperature and wind speed), the objects within the theater of operation (e.g., a high-rise building affecting wind flow or blocking view), and so on. The dispersal may be modeled using a Gaussian equation or other equations. As another example, if an incident is a release of radiation, the incident exercise system may calculate incident effects based on a model of radiation concentration at various locations considering factors such as amount and decay rate of the radioactive material, material within objects (e.g., lead), shielding by moving vehicles or intervening buildings, and so on. The amount and decay of radioactive material may be modeled using Bateman equations for the radioactive material.

During an incident exercise, the incident exercise system generates detector signals based on the incident effects that represent the detector signals the detection hardware of a detector would generate in an actual incident. Each detector may have a training mode in which the detector signals are received from the incident exercise system rather than the detection hardware of the detector. The incident exercise system then provides the detector signals to the detector for processing as if the detector had actually detected the effects of the incident. A detector with a mode to receive detector signals from an external source is described in U.S. Pat. No. 9,836,993, entitled “Realistic Training Scenario Simulations and Simulation Techniques” and issued on Dec. 5, 2017, which is hereby incorporated by reference.

During an incident exercise, the incident exercise system tracks the location of moving objects such as trainees holding detectors (e.g., walking or driving), detectors located on unmanned vehicles, and vehicles and participants moving through the theater of operation. The locations may be determined using the Global Positioning System (GPS), triangulation based on cellular signals or WiFi or other signals, cameras and image analysis, and so on given the current location of a detector, the incident exercise system models the combined effects of the incident effects of an incident and the characteristics effects of characteristics (e.g., radiation emitted by a manufacturing facility) of the theater of operation at that location. The incident exercise system then generates the detector signals that the detection hardware would generate given the combined effects.

Typically, a person using a detector seeks to assess only incident effects of the hazardous material incident and not characteristic effects resulting from characteristics of objects within the theater of operation (e.g., radiation emitted by non-hazardous content of a shipping container). Some detectors attempt to isolate the incident effects by identifying and filtering out “background noise” (e.g., background radiation). For example, a detector may process a moving window of detector signals generated by the detection hardware to generate a signature of the current background noise. The detector continually removes the current background noise from the total of detector signals resulting in filtered signals. The filtered signals represent the incident effects and possibly characteristic effects not considered to be background noise. The detector then generates an output based on the filtered signals.

The incident exercise system generates detector signals representing the combined effects so that the detector filters out background noise resulting in filter signals represent the incident effects and any characteristic effects not filtered out (e.g., not considered background noise). If a hazardous material incident has not yet occurred according to the exercise plan, the combined effects would represent just the characteristic effects resulting in the detector learning and filtering out the background noise and generating an output that the detector would generate given the characteristic effects of the characteristics of the theater of operation as defined by the exercise plan. If a detector in normal operation does not accurately filter out background noise effects, the detector generates a combined output which can reduce sensitivity for the incident effects but is the actual output the detector would generate given a real hazardous incident.

In some embodiments, the incident exercise system allows an exercise plan to be dynamically modified to account for unexpected activity in the theater of operation. For example, if the theater of operation is an active shipping facility that is processing shipments, objects (e.g., workers or shipping containers) moving about the facility would affect the detector signals generated by detection hardware, especially for moving shipments that contain background-impacting signals or shielding. When an incident commander notices the unexpected activity, the incident commander (controlling the overall incident exercise) can modify the exercise plan to indicate the presence of the unexpected (or otherwise unplanned) activity. If a container travels on a known route at a known speed, for example, from an entry portal to a container ship or a storage location, the exercise plan can include the route information so that the incident commander can modify the exercise plan by indicating the presence of the container and its route. As another example, the incident commander may want to provide a training scenario in which a trainee encounters a person who emits an abnormal amount of radiation such as after having radiation therapy to treat a cancer. As a delivery vehicle enters the shipping facility, the incident commander may modify the exercise plan so that the detector generates an output indicative of such abnormal amount of radiation and possibly radiation emitted by the container. Upon noticing an unusual amount of radiation, the trainee may be trained to request the driver to move away from the vehicle so that vehicle can be scanned separately from the driver to isolate the source of the abnormal amount of radiation. In such a case, the incident commander can direct the generation of detector signals to represent the radiation emitted by the container such as the radiation emitted by typical content of the container or by a hazardous material such as a dirty bomb. As another example, the exercise commander may notice that a trainee is not responding to a situation in an appropriate way. In such a case, the incident commander may direct the trainee to backtrack and how to respond. If the trainee responds correctly, the incident exercise may continue as planned.

The incident exercise system may also automatically modify the exercise plan based on images collected within the theater of operation. Cameras may be located throughout the theater of operation, located on vehicles (e.g., drones), worn by the trainee, and so on. The incident exercise system can then process the images to identify objects representing unexpected activity (e.g., arrival of a container) and track movement of the objects through the theater of operation. Various machine learning techniques may be used to process the images to identify the type of object and to track its current location. For example, a convolutional neural network (CNN) may be trained to identify containers, people, trains, and so on. Their locations may be identified based on the objects within the image (e.g., identified using a CNN), positions of the cameras (e.g., using triangulation), and so on.

The incident exercise system may also process the images to identify characteristics of the theater of operation not included in the exercise plan or characteristics encountered when the trainee moves outside the theater of operation. For example, the exercise plan may not specify all the buildings within the theater of operation. In such a case, when the building is detected, the incident exercise system may modify the exercise plan to account for the building. The incident exercise system may also process the images to identify the material that the building is made of such as wood, concrete, steel, brick, and so on. Once the material is identified, the incident exercise system can then generate detector signals based on the characteristics effects associated with the identified material. Various machine learning or other techniques may also be used to identify objects, their types (e.g., gas station and bridge) and materials, and so on. The incident commander may dynamically modify the exercise plan to specify characteristics of the identified objects.

The incident exercise system may employ various algorithms to model incident effects and characteristic effects. The algorithms may model explosion, fires, releases, plumes, resuspensions, and so on involving hazardous materials. The algorithms may factor in ground shine, wet and dry deposition, wind speed, and so on. Some algorithms for modeling hazardous materials depositions on the ground, for example, include code such as HotSpot as described in “HotSpots Healthy Physics Code—User's Guide,” National Atmospheric Release Advisory Center, Lawrence Livermore National Laboratory, LLNL-SM-63674, v. 3, Aug. 27, 2014, which is hereby incorporated by reference.

The incident exercise system may also support conducting an incident exercise that is partially or wholly virtual. When conducted virtually, a participant may wear a virtual reality or augmented reality headset. The incident exercise system may send to the headset images of the theater of operation corresponding to the current view of the participant. The incident exercise system may also send an image of a detector and/or its user interface assuming the participant is not holding a real detector or a virtual detector. A virtual detector may be a mockup of a real detector that displays an approximation of the output (e.g., generated by the incident exercise system) that a real detector would output. A virtual detector may also be used in a non-virtual incident exercise, for example, if real detectors are not available.

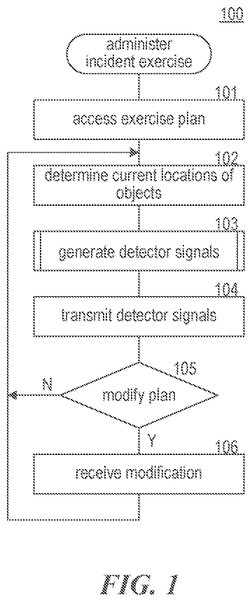

FIG. 1 is a flow diagram that illustrates the processing of an administer incident exercise component of the incident exercise system in some embodiments. The administer incident exercise component 100 is invoked to control the overall administration of an incident exercise based on an exercise plan. In block 101, the component accesses an exercise plan that specifies objects, detectors, incidents, and so on of the incident exercise. In blocks 102-106, the component loops generating detector signals based on current state of the incident exercise such as location of detectors, objects, and so on. In block 102, the component determines the current location of the objects that may be moving. In block 103, the component invokes a generate detector signals component to generate detector signals for each detector specified in the exercise plan. In block 104, the component transmits the detector signals to the detectors. In decision block 105, if the incident commander indicates to modify the exercise plan, then the component continues at block 106, else the component loops to block 102 to continue the incident exercise, for example, at the next time increment. In block 106, the component receives the modification to the exercise plan and then loops to block 102 to continue the incident exercise.

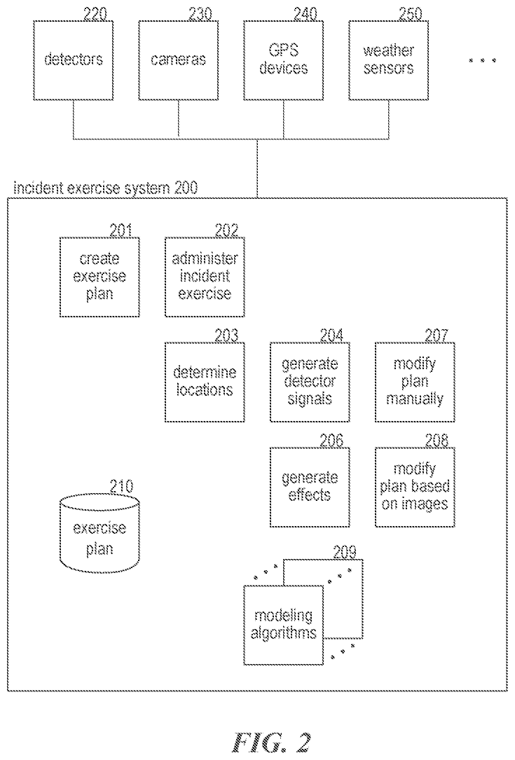

FIG. 2 is a block diagram that illustrates components of the incident exercise system in some embodiments. The incident exercise system 200 includes a create exercise plan component 201, an administer incident exercise component 202, a determine location component 203, a generate detector signals component 204, a generate effects component 206, a modify plan manually component 207, a modify plan based on images component 208, modeling algorithms 209, and an exercise plan data store 210. The incident exercise system communicates with detectors 220, cameras 230, GPS devices 240, the weather sensors 250, and so on. The incident exercise system may also communicate with other devices (e.g., smart phones and tablets) such as those held by the participants. The incident exercise system may provide information to the participants such as instructions from the incident commander, current state of the incident exercise, and so on and may receive responses from the participants (e.g., questions on how to handle a situation). The create exercise plan component provides a user interface that allows an incident exercise to be specified. The administer incident exercise component controls the overall conducting of the incident exercise. Th determine location component determines the location of objects, detectors, participants, and so on, for example, using GPS information, images, radar, and so on. The generate detector signals component generates detector signals for each detector based on the current state of the incident exercise. The generate effects component generates the incident effects and characteristics affects used to generate detector signals. The modify plan manually component allows an incident commander to modify the exercise plan dynamically during the incident exercise. The modify plan based on images component allows the exercise plan to be modified based on information extracted from the images. The modeling algorithms support modeling different types of incidents, for example, using HotSpots code. The exercise plan data stores the exercise plan.

The computing devices and systems on which the incident exercise system may be implemented may include a central processing unit, input devices, output devices (e.g., display devices and speakers), storage devices (e.g., memory and disk drives), network interfaces, graphics processing units, accelerometers, cellular radio link interfaces, global positioning system devices, and so on. The input devices may include keyboards, pointing devices, touch screens, gesture recognition devices (e.g., for air gestures), head and eye tracking devices, microphones for voice recognition, and so on. The computing devices may include desktop computers, laptops, tablets, e-readers, personal digital assistants, smartphones, gaming devices, servers, and computer systems such as massively parallel systems. The computing devices may access computer-readable media that include computer-readable storage media and data transmission media. The computer-readable storage media are tangible storage means that do not include a transitory, propagating signal. Examples of computer-readable storage media include memory such as primary memory, cache memory, and secondary memory (e.g., DVD) and include other storage means. The computer-readable storage media may have recorded upon or may be encoded with computer-executable instructions or logic that implements the incident exercise system. The data transmission media is used for transmitting data via transitory, propagating signals or carrier waves (e.g., electromagnetism) via a wired or wireless connection.

The incident exercise system may be described in the general context of computer-executable instructions, such as program modules and components, executed by one or more computers, processors, or other devices. Generally, program modules or components include routines, programs, objects, data structures, and so on that perform particular tasks or implement particular data types. Typically, the functionality of the program modules may be combined or distributed as desired in various embodiments. Aspects of the incident exercise system may be implemented in hardware using, for example, an application-specific integrated circuit (“ASIC”).

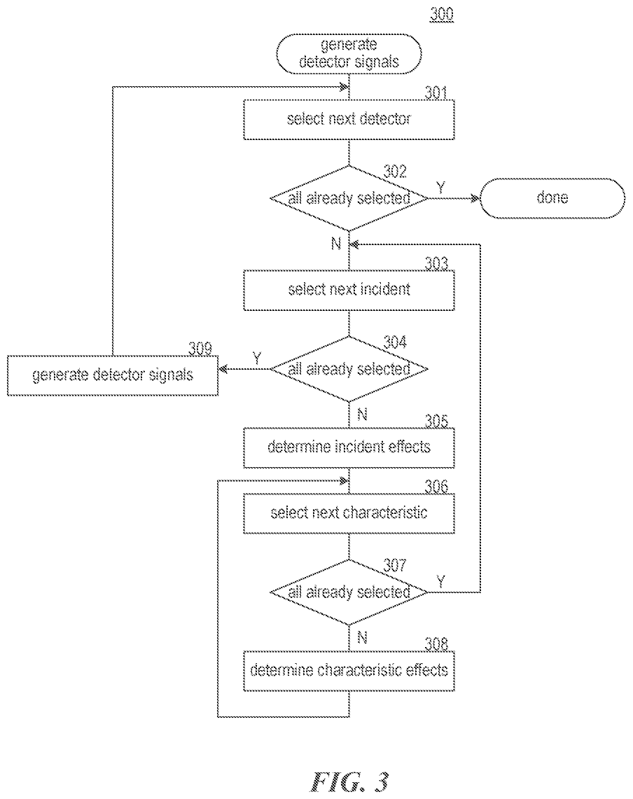

FIG. 3 is a flow diagram that illustrates the processing of a generate detector signals component of the incident exercise system in some embodiments. The generate detector signals component 300 controls the generating of detector signals based on current state of the incident exercise. In block 301, the component selects the next detector. In decision block 302, if all the detectors already been selected, then the component completes, else the component continues at block 303. In block 303, the component selects the next hazardous incident. In decision block 304, if all the hazards incidents have already been selected, then the component continues at block 309, else the component continues at block 305. In block 305, the component determines the incident effects of the selected incident on the selected detector. In block 306, the component selects the next characteristic within the theater of operation. In decision block 307, if all the characteristics have already been selected, then the component loops to block 303 to select the next incident, else the component continues at block 308. In block 308, the component determines the characteristic affects associated with the selected characteristic and then loops to block 306 to select the next characteristic. In block 309, the component generates detector signals for the selected detector based on the incident effects and characteristic effects and loops to block 301 to select the next detector.

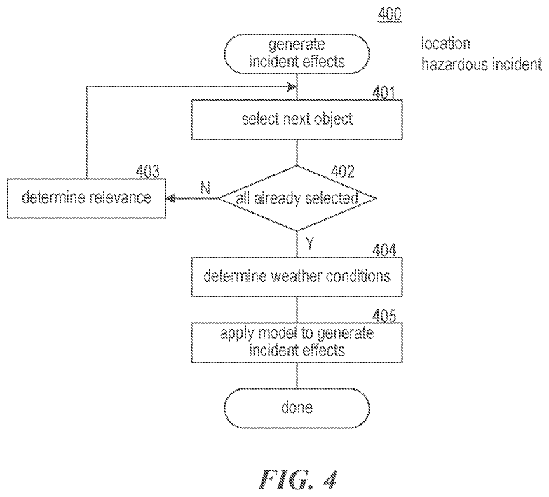

FIG. 4 is a flow diagram that illustrates the processing of a generate incident effects component of the incident exercise system in some embodiments. The generate incident affects component 400 is invoked passing an indication of the location of detector and the location of a hazardous incident in generates the corresponding incident effects the location of the detector. In block 401, the component selects the next object of the exercise plan. In decision block 402, if all the objects have already been selected, then the component continues at block 404, else the component continues at block 403. In block 403, the component determines the effect (if any) of the selected object (e.g., brick wall of building) on the incident effects and loops to block 401 to select the next object. In block 404, the component retrieves the weather conditions that impact the incident effects. In block 404, the component applies one or more algorithms to determine the incident effects at the location of the detector based on the weather conditions and effects of objects. The component then completes. The incident exercise system generates characteristic effects in a similar manner.

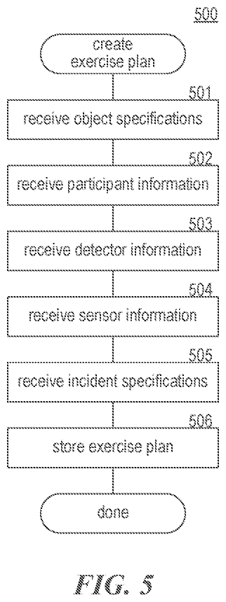

FIG. 5 is a flow diagram that illustrates the processing of a create exercise plan component of the incident exercise system in some embodiments. The create exercise plan component 500 is invoked to create an exercise plan. In block 501, the component receives specifications of objects (e.g., object type, size, location, and construction material) within the theater of operation. In block 502, the component receives participant information (e.g., name, role, and assigned detector). In block 503, the component receives detector information (e.g., detector type and location). In block 504, the component receives sensor information (e.g., type and location). In block 505, the component receives specifications of incidents (e.g., type, location, time, and shielding). In block 506, the component stores the information and specifications as an exercise plan in the exercise plan data store and completes.

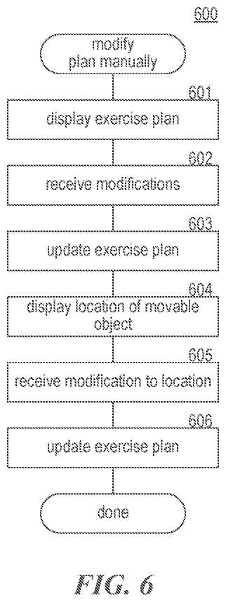

FIG. 6 is a flow diagram that illustrates the processing of a modify exercise plan manually component of the incident exercise system in some embodiments. The modify exercise plan manually component 600 is invoked during an incident exercise to modify the exercise plan based on input from an incident commander. In block 601, the component displays information relating to the exercise plan. In block 602, the component receives an indication of the modification (e.g., a specification of a new hazardous material incident and a new vehicle) from the incident commander. In block 603, the component updates the exercise plan based on the modification. In block 604, the component displays an indication of the locations of movable objects. In block 605, the component receives an indication of the modification to a location of a movable object. In block 606, the component updates the exercise plan based on the modification.

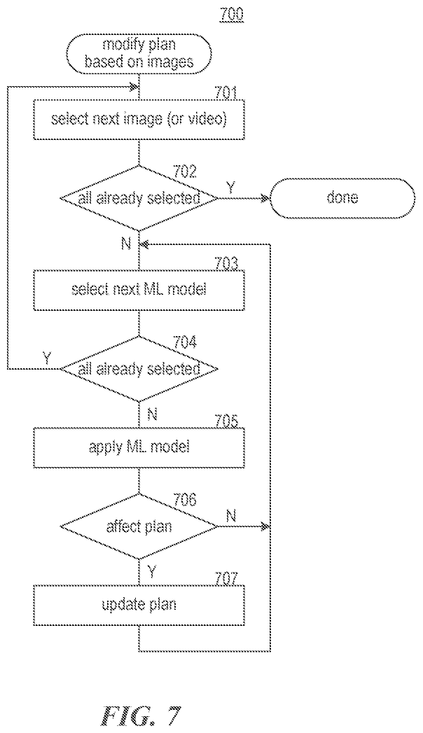

FIG. 7 is a flow diagram that illustrates the processing of a modify plan based on images component of the incident exercise system in some embodiments. The modify plan based on images component 700 is invoked to update the exercise plan based on analysis of images of the theater of operation. In block 701, the component selects the next image (or video clip). In decision block 702, if all the images have already been selected, then the component completes, else the component continues at block 703. In block 703, the component selects the next machine learning model that is trained to process images. In decision block 704, if all machine learning models have already been selected, then the component loops to block 701 to select the next image, else the component continues at block 705. In block 705, the component applies the selected learning model to the selected image. In decision block 706, if the machine learning model indicates that the exercise plan is affected (e.g., a vehicle is driven into the theater of operation), then the component continues at block 707, else the component loops to block 703 to select the next machine learning model. In block 707, the component updates the exercise plan and then loops to block 703 to select the next machine learning model.

The incident exercise system may use a variety of machine learning or techniques to process images, model incident effects and characteristic effects, modify an exercise plan dynamically, and so on. For example, a ML model may be trained to input a description of an incident (e.g., material, time, location) and a location and type of detector and output corresponding detector signals. Such a model may be used during an incident exercise rather than using a modeling algorithm to determine the incident effects and then generate detector signals. As another example, a ML model may be trained to input state an incident exercise and actions taken by a participant and output a modification to the exercise plan such as direct a truck to drive by the participant.

The ML techniques may include neural networks such as fully-connected, convolutional, recurrent, autoencoder, or restricted Boltzmann machine, a support vector machine, a Bayesian classifier, and so on. When a deep neural network is employed, the training results in a set of weights for the activation functions of the deep neural network.

A neural network model has three major components: architecture, cost function, and search algorithm. The architecture defines the functional form relating the inputs to the outputs (in terms of network topology, unit connectivity, and activation functions). The search in weight space for a set of weights that minimizes the objective function is the training process. In one embodiment, the classification system may use a radial basis function (“RBF”) network and a standard gradient descent as the search technique.

A convolutional neural network (CNN) has multiple layers such as a convolutional layer, a rectified linear unit (“ReLU”) layer, a pooling layer, a fully connected (“FC”) layer, and so on. Some more complex CNNs may have multiple convolutional layers, ReLU layers, pooling layers, and FC layers.

A convolutional layer may include multiple filters (also referred to as kernels or activation functions). A filter inputs a convolutional window, for example, of an image, applies weights to each pixel of the convolutional window, and outputs an activation value for that convolutional window. For example, if the image is 256 by 256 pixels, the convolutional window may be 8 by 8 pixels. The filter may apply a different weight to each of the 64 pixels in a convolutional window to generate the activation value also referred to as a feature value. The convolutional layer may include, for each filter, a node (also referred to a neuron) for each pixel of the image assuming a stride of one with appropriate padding.

The ReLU layer may have a node for each node of the convolutional layer that generates a feature value. The generated feature values form a ReLU feature map. The ReLU layer applies a filter to each feature value of a convolutional feature map to generate feature values for a ReLU feature map. For example, a filter such as max(0, activation value) may be used to ensure that the feature values of the ReLU feature map are not negative.

The pooling layer may be used to reduce the size of the ReLU feature map by downsampling the ReLU feature map to form a pooling feature map. The pooling layer includes a pooling function that inputs a group of feature values of the ReLU feature map and outputs a feature value.

The FC layer includes some number of nodes that are each connected to every feature value of the pooling feature maps.

The following paragraphs describe various embodiments of aspects of the incident exercise system. An implementation of the incident exercise system may employ any combination of the embodiments. The processing described below may be performed by a computing device with a processor that executes computer-executable instructions stored on a computer-readable storage medium that implements the incident exercise system.

In some embodiments, a method performed by one or more computing systems for administering an incident exercise is provided. The method accesses an exercise plan that the defines the incident exercise. The exercise plan specifies a type of the incident and a location and time of the incident within a theater of operation and specifying characteristics of objects within the theater of operation. The method receives a current location of a detector within the theater of operation at a current time. The method calculates incident effects of the incident at the current location and at the current time based on the exercise plan. The method calculates characteristic effects of the characteristics of the theater of operation at the current location and at the current time. The method generates detector signals based on a combination of the incident effects and the characteristic effects. The method sends to the detector the generated detector signals wherein the detector is adapted to filter effects of characteristics from detector signals to generate filtered effects of the incident and generate a user interface based on the filtered effects. In some embodiments, the incident is selected from a group consisting of a release of radiation, a release of a chemical, a seismic event, an atmospheric event, and an underwater event. In some embodiments, the incident is based on a release of radiation by a radioactive material and a characteristic of the theater of operation is background radiation. In some embodiments, the background radiation is based on an object within the theater of operation that emits radiation. In some embodiments, the object is a building. In some embodiments, the object is a walking surface. In some embodiments, the object is a container containing a non-hazardous content. In some embodiments, wherein the object is a person with a medical implant. In some embodiments, the object is a subsurface naturally occurring radioactive source.

In some embodiments, one or more computer-readable storage mediums storing computer-executable instructions for controlling one or more computing systems are provided. The instructions access an exercise plan that the defines an incident exercise within a theater of operation receive a current location of a detector within the theater of operation at a current time. The instructions calculate incident effects of the incident at the current location and at the current time based on the exercise plan. The instructions calculate characteristic effects of characteristics of the theater of operation at the current location and at the current time. The instructions detector signals based on a combination of the incident effects and the characteristic effects. The instructions send to the detector the generated detector signals. The detector is adapted to filter effects of characteristics from detector signals to generate filtered effects of the incident and generate a user interface based on the filtered effects.

In some embodiments, a method performed by one or more computing systems for dynamically adjusting an incident exercise is provided. The method accesses an exercise plan that the defines the incident exercise. The exercise plan specifies a type of the incident and a location and time of the incident within a theater of operation and specifying characteristics of the theater of operation. For each of a plurality of times during the incident exercise, the method determines incident effects of the incident at a current location based on the exercise plan, identifies a modification to the exercise plan, and updates the exercise plan based on the modification so that the incident continues with the updated exercise plan. In some embodiments, the updating includes adding an object to the theater of operation. In some embodiments, the updating includes resetting a current exercise time to an earlier time. In some embodiments, the incident is a radioactive incident and the updating affects a calculation of background radiation.

In some embodiments, one or more computing systems for administering an incident exercise are provided. The one or more computing systems includes one or more computer-readable storage mediums and one or more processors for executing instructions stored in the one or more computer-readable storage mediums. The one or more computer-readable storage mediums store an exercise plan that the defines the incident exercise. The exercise plan specifies objects within a theater of operation, specifies a type, a location, and a time of an incident within a theater of operation, specifies type and location of detectors within the theater of operation, and specifies characteristics of objects within the theater of operation. The one or more computer readable storage mediums store computer-executable instructions when executed by the one or more processors control the process to access a current location of a detector within the theater of operation at a current time, calculate incident effects of the incident at the current location and at the current time based on the exercise plan, calculate characteristic effects of the characteristics of the theater of operation at the current location and at the current time, generates detector signals based on a combination of the incident effects and the characteristic effects, and send to a detector the generated detector signals wherein the detector is adapted to filter effects of characteristics of the theater of operation from detector signals to generate filtered effects of the incident. In some embodiments, the incident is based on a release of radiation by a radioactive material and a characteristic of the theater of operation is background radiation. In some embodiments, the background radiation is based on an object within the theater of operation that emits radiation. In some embodiments, the computer-executable instructions identify objects based on analysis of images collected during the incident exercise. In some embodiments, the objects are identified using a machine learning model. In some embodiments, the computer-executable instructions modify the exercise plan during the incident exercise based on input from a person.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims. For example, the incident exercise system may be used with incidents that do not involve materials that are hazardous. For example, the incident may be related to an avalanche in a ski area with the incident exercise designed to train in locating avalanche victims using a sonic device or infrared detector. The incident exercise system may also be used in a gaming environment in which incidents involving hazardous materials (e.g., resulting from a dirty bomb) need to be identified and assessed so that counter measures can be taken. In such a gaming environment, the incident exercise system may generate the hazard data associated with an incident and provide the user experience for the detectors that are available to the participants in the game. Accordingly, the invention is not limited except as by the appended claims.

I/We claim:

1. A method performed by one or more computing systems for administering an incident exercise, the method comprising:
accessing an exercise plan that the defines the incident exercise, the exercise plan specifying a type of the incident and a location and time of the incident within a theater of operation and specifying characteristics of objects within the theater of operation;
receiving a current location of a detector within the theater of operation at a current time;
calculating incident effects of the incident at the current location and at the current time based on the exercise plan;
calculating characteristic effects of the characteristics of the theater of operation at the current location and at the current time;
generating detector signals based on a combination of the incident effects and the characteristic effects; and
sending to the detector the generated detector signals wherein the detector is adapted to filter effects of characteristics from detector signals to generate filtered effects of the incident and generate a user interface based on the filtered effects.

2. The method of claim 1 wherein the incident is selected from a group consisting of a release of radiation, a release of a chemical, a seismic event, an atmospheric event, and an underwater event.

3. The method of claim 1 wherein the incident is based on a release of radiation by a radioactive material and a characteristic of the theater of operation is background radiation.

4. The method of claim 3 wherein the background radiation is based on an object within the theater of operation that emits radiation.

5. The method of claim 4 wherein the object is a building.

6. The method of claim 4 wherein the object is a walking surface.

7. The method of claim 4 wherein the object is a container containing a non-hazardous content.

8. The method of claim 4 wherein the object is a person with a medical implant.

9. The method of claim 4 wherein the object is a subsurface naturally occurring radioactive source.

10. One or more computer-readable storage mediums storing computer-executable instructions for controlling one or more computing systems, the instructions comprising instructions to:
access an exercise plan that the defines an incident exercise within a theater of operation;
receive a current location of a detector within the theater of operation at a current time;
calculate incident effects of the incident at the current location and at the current time based on the exercise plan;
calculate characteristic effects of characteristics of the theater of operation at the current location and at the current time;
generate detector signals based on a combination of the incident effects and the characteristic effects; and
send to the detector the generated detector signals wherein the detector is adapted to filter effects of characteristics from detector signals to generate filtered effects of the incident and generate a user interface based on the filtered effects.

11. A method performed by one or more computing systems for dynamically adjusting an incident exercise, the method comprising:
accessing an exercise plan that the defines the incident exercise, the exercise plan specifying a type of the incident and a location and time of the incident within a theater of operation and specifying characteristics of the theater of operation; and
for a plurality of times during the incident exercise,
determining incident effects of the incident at a current location based on the exercise plan;
identifying a modification to the exercise plan; and
updating the exercise plan based on the modification so that the incident continues with the updated exercise plan.

12. The method of claim 11 wherein the updating includes adding an object to the theater of operation.

13. The method of claim 11 wherein the updating includes resetting a current exercise time to an earlier time.

14. The method of claim 11 wherein the incident is a radioactive incident and the updating affects a calculation of background radiation.

15. One or more computing systems for administering an incident exercise, the one or more computing systems comprising:
one or more computer-readable storage mediums for storing
an exercise plan that the defines the incident exercise, the exercise plan specifies objects within a theater of operation, specifies a type, a location, and a time of an incident within a theater of operation, specifies type and location of detectors within the theater of operation, and specifies characteristics of objects within the theater of operation; and
computer-executable instructions for controlling the one or more computing systems to:
access a current location of a detector within the theater of operation at a current time;
calculate incident effects of the incident at the current location and at the current time based on the exercise plan;
calculate characteristic effects of the characteristics of the theater of operation at the current location and at the current time;
generate detector signals based on a combination of the incident effects and the characteristic effects; and
send to a detector the generated detector signals wherein the detector is adapted to filter effects of characteristics of the theater of operation from detector signals to generate filtered effects of the incident; and
one or more processors for executing the computer-executable instructions stored in the one or more computer-readable storage mediums.

16. The one or more computing systems of claim 15 wherein the incident is based on a release of radiation by a radioactive material and a characteristic of the theater of operation is background radiation.

17. The one or more computing systems of claim 16 wherein the background radiation is based on an object within the theater of operation that emits radiation.

18. The one or more computing systems of claim 15 wherein the computer-executable instructions identify objects based on analysis of images collected during the incident exercise.

19. The one or more computing systems of claim 18 wherein the objects are identified using a machine learning model.

20. The one or more computing system of claim 14 wherein the computer-executable instructions modify the exercise plan during the incident exercise based on input from a person.
